(19) United States
(12) Patent Application Publication
(10) Pub. No.: US 2004/0176684 A1
(43) Pub. Date: Sep. 9, 2004

(54) Endoscope pretest capsule

(75) Inventors: Masafumi Tabuchi, Tokyo (JP); Shinji Takeuchi, Saitama-shi (JP); Shuichi Yamataka, Saitama-shi (JP)

Correspondence Address: Ronald R. Snider, P.O. Box 27613, Washington, DC, 20038-7613, US

(73) Assignees: Fuji Photo Optical Co., Ltd., Saitama-shi (JP); Masafumi TABUCHI, Tokyo (JP)

(21) Appl. No.: 10/781,944

(22) Filed: Feb. 20, 2004

(30) Foreign Application Priority Data
Feb. 21, 2003 (JP) 2003-44095

(51) Int. Cl. A61B 5/05

(52) U.S. Cl. 600/424; 128/899; 600/431

(57) ABSTRACT

An endoscope pretest capsule has approximately the same shape and weight as a capsule-type endoscope and is brought into the body before applying the capsule-type endoscope, and it includes barium sulfate which is a radiopaque substance (or a microcapsule wrapping a radiopaque substance) in a capsule body formed by a material having main components such as chitosan, and has an enteric coat for dissolving after elapse of a predetermined time provided on the surface side thereof. It is possible, by having the pretest capsule swallowed by an examinee in advance, to grasp passage conditions of the capsule-type endoscope in the body and secure good discharge out of the body because it dissolves in the case of remaining in the body for the predetermined time or longer.

### BACKGROUND OF THE INVENTION

The application claims the priority of Japanese Patent Applications No. 2003-44095 filed on Feb. 21, 2003 which is incorporated herein by reference.

1. Field of the Invention

The present invention relates to an endoscope pretest capsule to be swallowed before bringing a wireless capsule-type endoscope into the body in order to grasp in advance whether or not the endoscope can pass through the body well without remaining therein.

2. Description of the Related Art

There is a conventionally used flexible endoscope for imaging a desired region of a digestive organ and so on by inserting an end insert portion into an observed body, and an image thereby obtained can be observed on a monitor and so on. As opposed to such an endoscope, a capsule-type endoscope disclosed in Japanese Patent Laid-Open No. 2001-91860 or the like has been developed for the purposes of alleviating pain for a patient and so on.

FIG. 3 shows a block diagram as an example of the capsule-type endoscope. As shown therein, a transparent window portion 2 is mounted on the front side of a capsule proper 1. Inside the endoscope, right and left illumination LEDs (light-emitting diodes) 3A and 3B for emitting light via the transparent window portion 2, an objective 4 and an imaging device (CCD) 5 for shooting the region lit up by illumination light, and a circuit portion (board) 6 for processing an imaging signal outputted from the imaging device 5 and performing wireless communication between the capsule-type endoscope and an external (extracorporeal) device are provided, and a battery 7 is placed as a driving source for driving the circuit portion 6 and imaging device 5.

Such a capsule-type endoscope is brought into the body by having it swallowed by a patient having undergone a pretreatment such as restricted diet. In the endoscope brought into the body, each portion operates based on the power of the battery 7, and each region in transit is illuminated by the illumination LEDs 3A and 3B. The region is imaged by the objective 4 and imaging device 5 so that the imaging signal is sent to the external device outside the body via an antenna of the circuit portion 6. And in the external device, the received imaging signal is processed as a picture signal, and an image in the observed body shot by the capsule-type endoscope is displayed on the monitor and so on based on the picture signal.

### SUMMARY OF THE INVENTION

However, a capsule-type endoscope which is currently implemented does not have propulsion means for advancing on its own in the body and passively flows in a digestive organ. Therefore, there is a problem that it remains in the body if it cannot pass through the locations of constrictions, irregularities, lesions and so on existing in the digestive organ.

As described in FIG. 3, this type of capsule-type endoscope has a battery 7 mounted as a driving source therein. Therefore, there is a possibility that, if this endoscope remains in the body for a long time, there may arise an inconvenience such as outflow of the substance in the battery 7.

The present invention has been implemented in consideration of the above problem, and an object thereof is to provide an endoscope pretest capsule capable of being brought into the body as a test before actually using the capsule-type endoscope and grasping the state of passage of the capsule-type endoscope inside the body.

To attain the object, the endoscope pretest capsule (dummy capsule) according to the present invention is characterized by being brought into the body before applying the capsule-type endoscope, being in the shape (size) for grasping (examining) the state of passage of the capsule-type endoscope inside the body and dissolving in the case of remaining in the body for a predetermined time or longer.

The pretest capsule has a configuration in which an enteric coat is provided on a surface of the capsule body made of a material dissoluble in the body, for instance, so as to be decomposed and dissolve in the intestines. It is also possible to constitute the pretest capsule so that a radiopaque substance is put in a fluid-insoluble microcapsule and the microcapsule is placed inside the capsule body made of the material dissoluble in the body.

According to the above configuration, the pretest capsule is formed by an enteric (intestine-dissoluble) material which dissolves after a predetermined time (24 hours for instance) as the one having approximately the same shape, size and weight as the capsule-type endoscope, for instance. For instance, as a method of producing it, the capsule body is formed by the material of which main component is chitosan, a natural polymer, and an alkali soluble enteric coat such as (acetic acid/succinic acid) hydroxi-propyl-methylcellulose is affixed on the surface thereof. According to the pretest capsule, it is possible to determine whether or not the passage through the digestive organ is feasible by checking discharge out of the body. And if it remains in the body, the capsule dissolves in the body due to elapse of 24 hours or longer and is easily discharged out of the body.

In the case where the radiopaque substance is included in the pretest capsule, it is possible to check and grasp the state of passage of the capsule in detail by observing the existence of the capsule with a fluoroscope.

### BRIEF DESCRIPTION OF THE DRAWINGS

FIG. 1 is a sectional view showing a configuration of an endoscope pretest capsule according to a first embodiment of the present invention;

FIGS. 2A and 2B show the configuration of the endoscope pretest capsule according to a second embodiment, where FIG. 2A is a sectional view thereof and FIG. 2B is a sectional view of a microcapsule inside it; and

FIG. 3 is a perspective view showing an overview configuration of a proposed capsule-type endoscope.

### DETAILED DESCRIPTION OF THE PREFERRED EMBODIMENTS

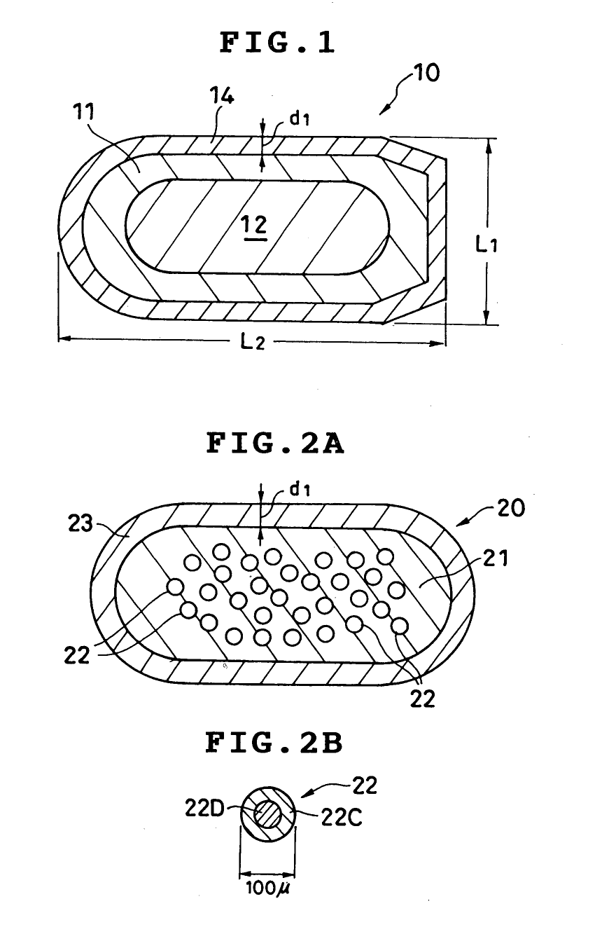

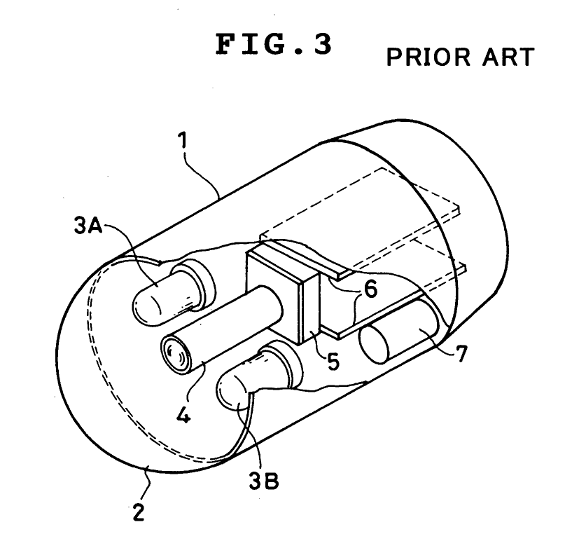

FIG. 1 shows a sectional view of an endoscope pretest capsule according to a first embodiment. A pretest capsule (capsule body) 10 of this embodiment is formed to have the same shape, size and weight as the capsule-type endoscope in FIG. 3, and has a vertical length (diameter) of approximately 10 mm and a horizontal length of approximately 20 mm for instance. To be more specific, the shape, size and weight of the pretest capsule 10 are set on a condition for checking and grasping a state of passage in the body. They should be approximately the same or similar figures with slight differences in size and weight.

The pretest capsule 10 has a configuration in which barium sulfate (liquid or solid) 12 as a radiopaque substance is included in a capsule body 11 formed by a material having main components of chitosan, gelatin, cellulose and so on of natural polymers (fluid-soluble material), and an alkali soluble enteric coat 14 such as (acetic acid/succinic acid) hydroxi-propyl-methylcellulose is provided on the surface thereof. The enteric coat 14 is set at dissolution time of 24 hours or longer by appropriately selecting its material, thickness and so on. The dissolution time is determined to suit internal residence time of one to seven days (one week). It is also possible to produce a plurality of the pretest capsule 10 of which dissolution time in the body is different so as to appropriately select and use them according to circumstances.

According to such a configuration of the first embodiment, the pretest capsule 10 is swallowed by an examinee before using the capsule-type endoscope described in FIG. 3. As the pretest capsule 10 is discharged (excreted) out of the body after 24 hours or so under normal circumstances, it is possible to check the state of the passage of the capsule-type endoscope by checking the discharge out of the body. If the inside of the body of the examinee is seen through with a fluoroscope in this case, it is possible to check the passage position of the pretest capsule 10 in the body by having the barium sulfate 12 in one's sight so as to examine and grasp in detail the passage conditions of the pretest capsule 10 such as a situation of remaining in constrictions, irregularities, lesions and so on in the digestive organ and a remaining position thereof.

In the case where the pretest capsule 10 remains in the body and the time of 24 hours or longer has elapsed since it was taken in, the pretest capsule 10 itself dissolves in the intestines and so on and is discharged out of the body. To be more specific, the pretest capsule 10 dissolves and decomposes so as to resolve the state of remaining and be discharged out of the body. It is possible., by such an examination of the pretest capsule 10, to ensure the examination with the capsule-type endoscope and also prevent adverse effects due to long-time internal residence of the capsule-type endoscope.

FIG. 2 show the configuration of a second embodiment in which the radiopaque substance is put in a fluid-insoluble microcapsule and is placed. As shown in FIG. 2A, a pretest capsule 20 of the second embodiment is formed as a capsule as if a capsule body 21 formed by the material having chitosan, for instance, as its main component includes microcapsules 22. And an enteric coat 23 of d₁ thickness made of hydroxi-propyl-methylcellulose or the like is formed on the surface side of the capsule body 21.

And as shown in FIG. 2B, the microcapsule 22 is the size of 100 μm or so in diameter, and is formed by putting a radiopaque substance 22D made of metal and barium sulfate and so on inside a capsule body 22C of a generally used fluid (internal)-insoluble material.

According to such a configuration of the second embodiment, it is possible to grasp and check the state of the passage of the capsule-type endoscope of the same shape and weight by swallowing the pretest capsule 20 as in the case of the first embodiment. The pretest capsule 20 dissolves if the time of one to seven days elapses. If it remains in the body for a long time, it is decomposed into a melt and individual microcapsules 22 so as to be excreted out of the body. It is possible, by applying the fluoroscope to the examinee, to check in detail the passage conditions, situation of remaining in constrictions and irregularities, remaining position and so on of the pretest capsule 20 in the body due to the existence of the radiopaque substance 22D in the microcapsules 22.

According to the first and second embodiments, the radiopaque substances 12 and 22D are accommodated inside. It is possible, however, to constitute it only with the capsule bodies 11, 21 and the enteric coats 14, 23.

As described above, the present invention is constituted to provide the endoscope pretest capsule in approximately the same shape as the capsule-type endoscope to be brought into the body before applying the capsule-type endoscope, and dissolve the capsule itself in the case of remaining in the body for the predetermined time or longer. Therefore, there is an advantage that it is possible, by bringing it into the body as a test before using the capsule-type endoscope, to grasp the state of passage of the actual capsule-type endoscope inside the body so as to eliminate the adverse effects of the battery and so on in the case where the capsule-type endoscope remains.

It is also possible, by including the radiopaque substance in the pretest capsule, to detailedly check and grasp the passage conditions, situation of remaining in constrictions, irregularities and lesions, remaining position and so on of the pretest capsule in the body.

What is claimed is:

1. An endoscope pretest capsule, wherein it is formed to be brought into the body before applying a capsule-type endoscope, have a shape for grasping a state of passage of the capsule-type endoscope inside the body and dissolve in the case of remaining in the body for a predetermined time or longer.

2. The endoscope pretest capsule according to claim 1, wherein it is constituted by an enteric material.

3. The endoscope pretest capsule according to claim 2, wherein it is constituted by providing an enteric coat on a surface of a capsule body made of a material dissoluble in the body.

4. The endoscope pretest capsule according to claim 1, wherein it is comprised of a plurality of types constituted so that dissolution time in the body is different.

5. The endoscope pretest capsule according to claim 1, wherein it includes a radiopaque substance.

6. The endoscope pretest capsule according to claim 5, wherein the radiopaque substance is put in a fluid-insoluble microcapsule and the microcapsule is placed inside a capsule body made of a material dissoluble in the body.
